(19) United States
(12) Patent Application Publication
(10) Pub. No.: US 2010/0076265 A1
(43) Pub. Date: Mar. 25, 2010

(54) ENDOSCOPE FLEXIBLE SECTION AND ENDOSCOPE

(75) Inventors: Shinichi YAMAKAWA, Kanagawa (JP); Toshiaki FUKUNAGA, Kanagawa (JP); Shigeru NAKAMURA, Kanagawa (JP); Takayuki NAKAMURA, Kanagawa (JP)

Correspondence Address: Solaris Intellectual Property Group, PLLC, 401 Holland Lane, Suite 407, Alexandria, VA, 22314, US

(73) Assignee: FUJIFILM CORPORATION, Tokyo (JP)

(21) Appl. No.: 12/561,289

(22) Filed: Sep. 17, 2009

(30) Foreign Application Priority Data
Sep. 25, 2008 (JP) 2008-245475

(51) Int. Cl. A61B 1/005

(52) U.S. Cl. 600/139

(57) ABSTRACT

There is provided an endoscope and an endoscope flexible section that can secure bending rigidity and have the resistance against high temperature-and-pressure steam during sterilization. The endoscope flexible section includes a spiral tube, a net-like tube, and an outer cover member. The spiral tube is formed by winding a belt-like member made of metal, in a spiral shape. The net-like tube is formed in an annular shape on the periphery of the spiral tube by braiding thin metal wires. The outer cover member is formed on the periphery of the net-like tube. In the outer cover member, a polyparaxylene resin layer is formed in a pattern on the periphery of a cover layer that covers the periphery of the net-like tube and is made of fluororubber. The polyparaxylene resin layer is a pattern of the plural rings that are disposed in a circumferential direction at predetermined intervals.

### CROSS-REFERENCE TO RELATED APPLICATION

This application claims priority under 35 USC 119 from Japanese Patent Application No. 2008-245475 filed on Sep. 25, 2008, the disclosure of which is incorporated by reference herein.

### BACKGROUND OF THE INVENTION

1. Field of the Invention

The present invention relates to a tubular endoscope flexible section, and an endoscope including the endoscope flexible section.

2. Related Art

A long insertion section of a medical endoscope is inserted into a patient's body cavity so that the medical endoscope is used to observe internal organs. Alternatively, various medical treatments or actions are performed using a treatment tool that is inserted into a treatment tool insertion channel of the endoscope. For this reason, when the endoscope having been used once is used for another patient, the endoscope needs to be disinfected or sterilized after the completion of medical examination and actions in order to prevent the infection between patients through the endoscope. For disinfection or sterilization, there may be used a method using an autoclave or the like, which is a sterilization method using high temperature-and-pressure steam, or using disinfectant, ethylene oxide gas, formalin gas, hydrogen peroxide gas, plasma, or ozone.

A method, which uses an autoclave for sterilizing an endoscope with high temperature-and-pressure steam, is a sterilization method that has been widely used conventionally. This method has many merits, such as high reliability of a sterilizing effect, no residual toxicity, and low running cost. As a typical condition when an endoscope is sterilized by high temperature-and-pressure steam, American Standard ANSI/AAMI ST37-1992, which is approved by American National Standards Institute (ANSI) and issued by Association for the Advancement of Medical Instrumentation, discloses that a sterilizing process where a temperature of 132° C. and 4 minutes in the case of a pre-vacuum type method and a sterilizing process where a temperature of 132° C. and 10 minutes in the case of a gravity type method. However, the damage applied to medical instrumentation is significantly serious under such environment.

A soft section (a flexible tube) of an insertion section of an endoscope includes, for example, a spiral tube that is formed by winding a belt-like member in a spiral shape so as to have a constant diameter, a net-like tube that is formed on the outer periphery of the spiral tube by braiding thin wires, and an outer layer that covers the outer periphery of the net-like tube. If the net-like tube is formed by braiding stainless steel wires, the outer layer is generally made of a thermoplastic resin material (thermoplastic elastomer). However, since the thermoplastic resin material is thermally deformed by the heating of a process using the autoclave, there is a possibility that the soft section is deformed to be “bent” (reformed to be bent) if the soft section (flexible tube) is put in the autoclave device in a state in which it is bent.

As a countermeasure against it, Japanese Patent No. 3833879 discloses an endoscope device in which a positioning section is provided in an endoscope aid that receives the endoscope. The positioning section restricts a predetermined portion of the soft section (flexible tube) so that the predetermined portion of the soft section is substantially straight during high pressure steam sterilization.

However, the endoscope device disclosed in Japanese Patent No. 3833879 requires a large-sized autoclave device in order to restrict a predetermined portion of the soft section (flexible tube) so that the predetermined portion of the soft section is substantially straight.

Further, as another countermeasure against “bent”, it may be considered that a material having the resistance against high temperature-and-pressure steam is used for the outer layer of the soft section (flexible tube). However, since the soft section requires flexibility and appropriate bending rigidity, it is difficult to select the material for the soft section. That is, since the soft section is a section that is inserted into a body cavity or the like, it is necessary for the soft section to have high flexibility in a bending direction. Meanwhile, the soft section needs to have appropriate rigidity against bending in order to receive thrust when the soft section is inserted into a body cavity or the like. Accordingly, it is important for the soft section to have the desired flexibility and bending rigidity in terms of insertion operability or the release of patient's pain.

### SUMMARY OF THE INVENTION

In consideration of the fact, the present invention provides an endoscope and an endoscope flexible section that can secure bending rigidity and have the resistance against high temperature-and-pressure steam during sterilization.

According to a first aspect of the invention, there is provided an endoscope flexible section that includes: a longitudinal tubular body; and an outer covering member formed on an outer periphery of the tubular body, the outer covering member including: a cover layer that covers the outer periphery of the tubular body and is made of fluororubber or silicone rubber; and a polyparaxylene resin layer formed in a pattern, disposed on an outer periphery of the cover layer or disposed inside the cover layer, so as to be discontinuous linearly in a longitudinal direction of the outer covering member.

According to the first aspect of the invention, the outer cover member is formed on the outer periphery of the long tubular body. In the outer cover member, a polyparaxylene resin layer is formed in a pattern on the outer periphery of a cover layer, which is made of fluororubber or silicone rubber, or inside the cover layer. The polyparaxylene resin layer is formed in a pattern that is linearly discontinuous in a longitudinal direction. The cover layer, which is made of fluororubber or silicone rubber, has the resistance against high temperature-and-pressure steam (resistance during the sterilization of an autoclave). However, since the cover layer is made of only fluororubber or silicone rubber, the rigidity of the cover layer is low. For this reason, in the invention, it may be possible to secure the bending rigidity of the endoscope flexible section by providing the outer cover where the polyparaxylene resin layer is formed in a pattern on the periphery of the cover layer or inside the cover layer. That is, the rigidity of fluororubber or the like is about a one-severalth to a one-tenth of that of a polyurethane resin that is a material of the outer cover of an existing endoscope flexible section. However, the rigidity of the polyparaxylene resin is several hundreds times larger than that of fluororubber or the like. Accordingly, if the pattern of the polyparaxylene resin is formed on the periphery of the cover layer made of fluororubber or inside the cover layer, it may be possible to increase the rigidity of the entire outer cover member up to the rigidity of the same level of a polyurethane resin. Therefore, it may be possible to obtain an endoscope flexible section that has the resistance against high temperature-and-pressure steam (resistance during the sterilization of an autoclave) and secures bending rigidity.

According to a second aspect of the invention, in the endoscope flexible section of the first aspect of the invention, the pattern of the polyparaxylene resin layer is a plurality of rings, each formed in a circumferential direction of the outer covering member, that are arranged in the longitudinal direction of the outer covering member, or the pattern of the polyparaxylene resin layer is a continuous spiral formed along the longitudinal direction of the outer covering member.

According to the second aspect of the invention, since the pattern of the polyparaxylene resin layer is the plural rings that are formed in a circumferential direction or a continuous spiral, it may be possible to secure desired bending rigidity while maintaining the flexibility of the endoscope flexible section.

According to a third aspect of the invention, in the endoscope flexible section of the second aspect of the invention, a bending rigidity of the outer covering member is changed in the longitudinal direction by changing a density or a pitch of the pattern of the polyparaxylene resin layer in the longitudinal direction. In the aspect, it is possible that a bending rigidity of the outer covering member is changed in the longitudinal direction by changing a pitch of the rings of the polyparaxylene resin layer in the longitudinal direction. Further, in the aspect, it is possible that a bending rigidity of the outer covering member is changed in the longitudinal direction by changing a pitch of the spiral of the polyparaxylene resin layer in the longitudinal direction. Moreover, it is possible that a density or a pitch of the pattern of the polyparaxylene resin layer at one end side in the longitudinal direction of the outer covering member is different from that at another side.

According to the third aspect of the invention, the bending rigidity of the endoscope flexible section is changed in the longitudinal direction by changing the density or pitch of the pattern of the polyparaxylene resin layer in the longitudinal direction. Accordingly, it may be possible to change the bending rigidity of a portion where it is necessary to change the bending rigidity of the endoscope flexible section with respect to the bending rigidity of another portion of the endoscope flexible section in the longitudinal direction by simple structure.

According to a fourth aspect of the invention, in the endoscope flexible section of any one of the first to third aspects of the invention, the thickness of the polyparaxylene resin layer may be from 5 μm to 80 μm.

According to the fourth aspect of the invention, the thickness of the polyparaxylene resin layer is from 5 μm to 80 μm. Accordingly, it may be possible to secure bending rigidity while maintaining the flexibility of the endoscope flexible section. In contrast, if the thickness of the polyparaxylene resin layer is smaller than 5 μm, it is difficult to secure desired bending rigidity. Further, if the thickness of the polyparaxylene resin layer is larger than 80 μm, it is difficult to maintain the flexibility of the endoscope flexible section.

According to a fifth aspect of the invention, in the endoscope flexible section of the first aspect of the invention, the tubular body includes a spiral tube that is formed by winding a belt-like member in a spiral shape, and a net-like tube that is provided on the outer periphery of the spiral tube and formed in an annular shape by braiding thin wires.

According to the fifth aspect of the invention, the tubular body includes the spiral tube that is formed by winding the belt-like member in spiral shape, and the net-like tube that is provided on the outer periphery of the spiral tube and formed in an annular shape by braiding thin wires. The outer cover member is formed on the outer periphery of the net-like tube. In the outer cover member, the polyparaxylene resin layer is formed in a predetermined pattern on the outer periphery of a cover layer member, which is made of fluororubber or silicone rubber, or inside the cover layer member. Accordingly, the endoscope flexible section may have the resistance against high temperature-and-pressure steam (resistance during the sterilization of an autoclave), and it may be possible to more effectively secure the bending rigidity of the endoscope flexible section.

According to a sixth aspect of the invention, there is provided an endoscope including a long insertion section to be inserted into a patient's body cavity including the endoscope flexible section of any one of the first to fifth aspects of the invention.

According to the sixth aspect of the invention, the long insertion section to be inserted into a patient's body cavity including the endoscope flexible section of any one of the first to fifth aspect of the invention. Accordingly, the endoscope flexible section may have the resistance against high temperature-and-pressure steam (resistance during the sterilization of an autoclave), and it may be possible to secure the bending rigidity of the endoscope flexible section.

According to the aspects of the invention, the endoscope flexible section may have the resistance against high temperature-and-pressure steam (resistance during the sterilization of an autoclave), and it may be possible to secure the bending rigidity of the endoscope flexible section.

### BRIEF DESCRIPTION OF THE DRAWINGS

Exemplary embodiments of the invention will be described in detail with reference to the following figures, wherein:

FIG. 1 is a schematic view showing the configuration of an endoscope using an endoscope flexible section according to a first exemplary embodiment of the invention;

FIG. 2 is an exploded side view of a part of the endoscope flexible section according to the first exemplary embodiment of the invention;

FIG. 3 is a cross-sectional view of the endoscope flexible section according to the first exemplary embodiment of the invention;

FIG. 4 is an exploded perspective view of a part of the endoscope flexible section according to the first exemplary embodiment of the invention;

FIG. 5 is an enlarged cross-sectional view of an outer cover of the endoscope flexible section according to the first exemplary embodiment of the invention;

FIG. 6 is a perspective view of an outer cover of an endoscope flexible section according to a second exemplary embodiment of the invention; and

FIG. 7 is an enlarged cross-sectional view of an outer cover of an endoscope flexible section according to a third exemplary embodiment of the invention.

### DETAILED DESCRIPTION OF THE INVENTION

Exemplary embodiments of the invention will be described with reference to drawings.

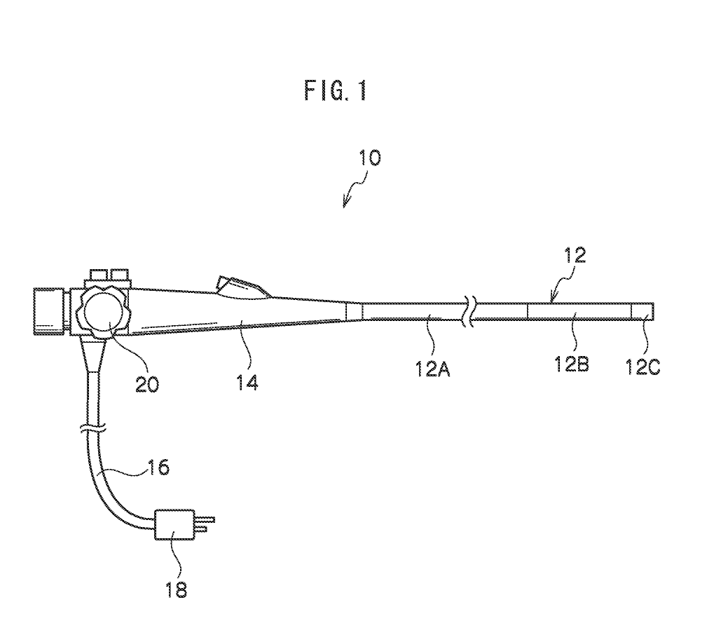

FIG. 1 shows the entire configuration of an endoscope 10 according to a first exemplary embodiment of the invention. As shown in FIG. 1, the endoscope 10 includes a long insertion section 12 that is inserted into a patient's body cavity, and a main body operation section 14 is connected to a base end portion of the insertion section 12. A long light guide soft section 16, which is detachably connected to a light source device (not shown in the drawings), is connected to the main body operation section 14. A connection section 18 including a terminal, which is connected to the light source device (not shown in the drawings), is provided at a tip end of the light guide soft section 16. Further, the main body operation section 14 is provided with an operation knob 20 that is used to operate the insertion section 12.

The insertion section 12 includes a flexible tube portion 12A as an endoscope flexible section, an angle portion 12B, and an end body 12C. The flexible tube portion 12A extends from a portion, which is connected to the main body operation section 14, in the longitudinal direction (axial direction), and forms a portion corresponding to most length of the insertion section. The angle portion 12B is connected at the tip end side of the flexible tube portion 12A in the longitudinal direction of the flexible tube portion. The end body 12C is connected at the tip end of the angle portion 12B in the longitudinal direction of the angle portion, and includes an objective optical system or the like therein. The angle portion 12B is remotely controlled to be bent by rotating the operation knob 20 that is provided at the insertion section 12. Further, the light guide soft section 16 has substantially the same structure as the flexible tube portion 12A of the insertion section 12.

The length of the flexible tube portion 12A is set to a length such that it secures a length enough for the end body 12C to reach the inside of a predetermined object to be observed, further, it allows an operator to be away from a patient or the like so that inconvenience is not occurred when the operator grips and operates the main body operation section 14. The flexible tube portion 12A needs to have flexibility over the entire length thereof. In particular, a portion of the flexible tube portion 12A, which is to be inserted into a patient's body cavity or the like, has higher flexibility. Further, the flexible tube portion 12A needs to have a predetermined rigidity against bending particularly at the portion thereof that is connected to the main body operation section 14, in order to obtain thrust when the flexible tube portion 12A is inserted into the body cavity or the like. Furthermore, the flexible tube portion 12A preferably has higher flexibility particularly at the portion thereof that is connected to the angle portion 12B so that the connected portion follows the curved shape of the angle portion 12B to some extent when the angle portion 12B is curved.

A light guide, an image guide (a signal cable in the case of an electronic endoscope), a treatment tool insertion channel, air and water send tubes, and the like (not shown in the drawings) are built inside a tubular portion of the flexible tube portion 12A.

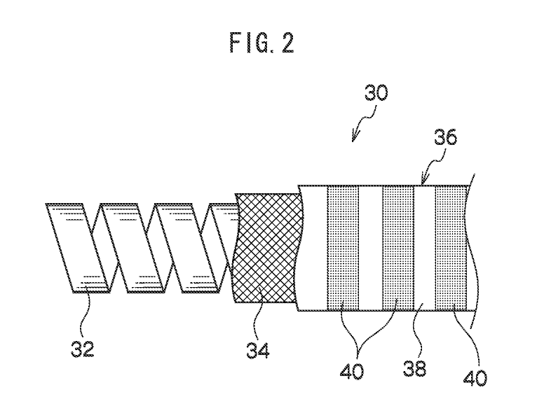

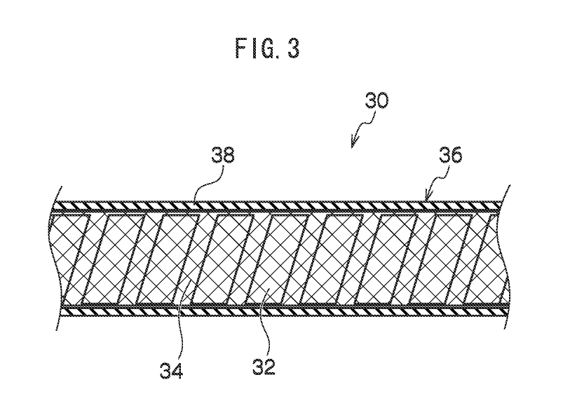

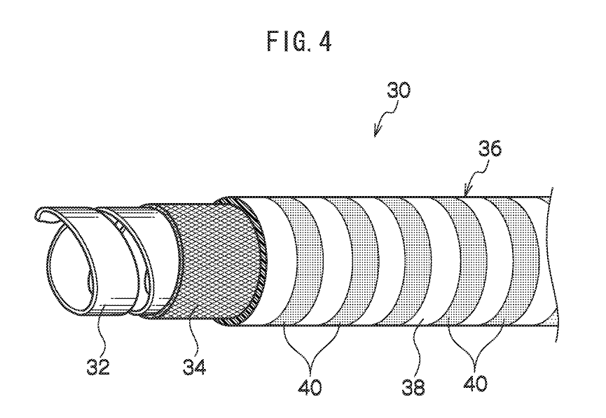

FIG. 2 is a partially cut side view of an endoscope flexible section 30 that forms the tubular portion of the flexible tube portion 12A. Further, FIG. 3 is a cross-sectional view of the endoscope flexible section 30, and FIG. 4 is a partially cut perspective view of the endoscope flexible section 30. As shown in these drawings, the endoscope flexible section 30 includes a spiral tube 32, a net (mesh)-like tube 34, and an outer cover member 36. The spiral tube 32 is formed by winding a belt-like member, which is made of metal, in a spiral shape. The net-like tube 34 is formed in an annular shape on the outer periphery of the spiral tube 32 by braiding thin metal wires (wire rods). The outer cover member 36 is formed on the outer periphery of the net-like tube 34. The spiral tube 32 and the net-like tube 34 formed on the outer periphery of the spiral tube 32 configure a “tubular body” of the invention.

The outer cover member 36 is formed by forming a polyparaxylene resin layer 40 in pattern (in a predetermined shape(s)) on the outer periphery of a cover layer 38 that covers the outer periphery of the net-like tube 34 and is made of fluororubber. As shown in FIGS. 2 and 4, the pattern of the polyparaxylene resin layer 40 is plural rings which are arranged at predetermined interval therebetween and each of which is formed along a circumferential direction.

A method of forming the outer cover member 36 will be described below.

The cover layer 38 made of fluororubber is formed on the outer periphery of the net-like tube 34. For example, the cover layer 38 may be formed in the shape of a tube on the outer periphery of the net-like tube 34, which is formed on the spiral tube 32, by an extrusion molding machine. In addition, the surface of the fluororubber is modified by atmospheric-pressure plasma. After that, a primer treatment is performed by using a silane coupling agent, it is put in the chamber of a chemical vapor deposition apparatus (not shown in the drawings) and the chamber is decompressed. Then, vaporized (gasified) diparaxylene is supplied to the chamber through a heating tube and it is left (put) under this atmosphere for a predetermined time, so that a polyparaxylene resin layer is formed. That is, for example, the chemical vapor deposition of a polyparaxylene resin is performed.

Examples of a polyparaxylene resin include polymonochloro-para-xylylene, polyparaxylene, polydichloro-para-xylylene, and polyfluorinated paraxylene. Accordingly, those (it) having appropriate properties (property) are (is) selected or combined and used as a coating agent. Coating thickness may be adjusted by changing left-time (put-time) under the resinous atmosphere. In order to form the pattern of the polyparaxylene resin, portions, which do not need to be coated, on the cover layer 38, are covered by a mask, the chemical vapor deposition of polyparaxylene is performed on the cover layer 38, and then the mask is removed. Accordingly, the pattern of the polyparaxylene resin layer 40 is formed. The thickness of the polyparaxylene resin layer 40 (coating thickness) is preferably in the range of 5 to 80 μm, and more preferably in the range of 10 to 30 μm. The lower limit of the thickness of the polyparaxylene resin layer 40 is set in order to secure the bending rigidity of the endoscope flexible section 30, and the upper limit of the thickness of the polyparaxylene resin layer 40 is set in order to maintain the flexibility of the endoscope flexible section 30. That is, if the thickness of the polyparaxylene resin layer 40 is smaller than 5 μm, it is difficult to secure desired bending rigidity. Further, if the thickness of the polyparaxylene resin layer is larger than 80 μm, it is difficult to maintain the flexibility of the endoscope flexible section.

The rigidity of fluororubber is about a one-severalth to a one-tenth of that of a polyurethane resin that is a material of the cover of the endoscope flexible section of the existing or conventional endoscope. However, the rigidity of a polyparaxylene resin is several hundreds times larger than that of fluororubber. Accordingly, if the pattern of the polyparaxylene resin layer 40 is formed on the cover layer 38 that is made of fluororubber, it may be possible to increase the rigidity of the outer cover member 36 as a whole up to substantially the same level of the rigidity of a polyurethane resin. That is, in this exemplary embodiment, the cover layer 38 made of fluororubber is reinforced by the polyparaxylene resin layer 40.

As shown in FIG. 4, the pattern, that is, plural rings of the polyparaxylene resin layer 40 is formed on the cover layer 38 made of fluororubber. It may be possible to change the bending rigidity of the outer cover member 36 along the longitudinal direction of the endoscope flexible section 30 by changing the density of the pattern or pitch of the pattern (pitch of the plural rings for example in FIG. 4) of the polyparaxylene resin layer 40, which is formed on the cover layer 38, along the longitudinal direction (axial direction).

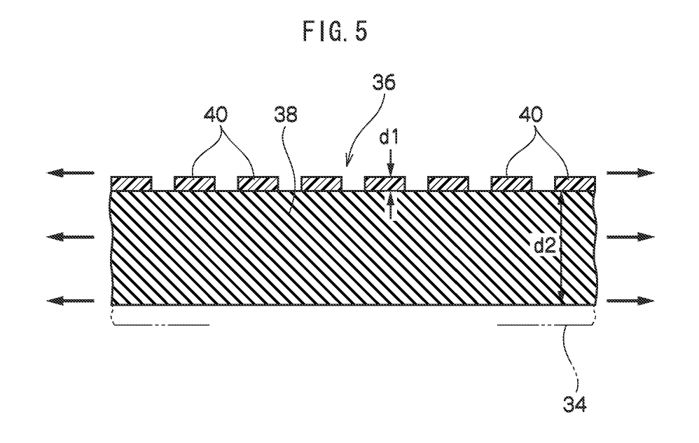

FIG. 5 is a cross-sectional view of the pattern of the polyparaxylene resin layer 40 that is of the plural rings and is formed on the cover layer 38. For example, if the polyparaxylene resin layer 40 covers about 80% of the surface of the cover layer 38 made of fluororubber, when the endoscope flexible section 30 is formed by the materials shown in Table 1 in the conditions of rigidity and thickness of respective materials of the cover layer 38 and the polyparaxylene resin layer 40 shown in Table 1, it may be possible to obtain the same rigidity as the outer cover member of the endoscope that is made of polyurethane. In this exemplary embodiment, Parylene C (manufactured by Parylene Japan, Inc.) is used for the polyparaxylene resin layer 40.

TABLE 1

| | Modulus of elasticity [MPa] | Thickness [mm] |
|---|---|---|
| Fluororubber | 5.92 | d2 = 0.1 |
| Polyparaxylene resin | 2.76 × 10³ | d1 = 0.02 |

In this case, it has been known that the yield strain of a polyparaxylene resin is about 3%. The strain of the polyparaxylene resin layer 40 should be smaller than 3% in order to prevent the polyparaxylene resin layer 40 from yielding when the endoscope flexible section 30 is curved. In general, when the outer cover member of the endoscope flexible section is curved, strain of about 20% is generated at the outer cover member of the endoscope flexible section. It is understood as follows: if the polyparaxylene resin layer 40 covers 80% of the surface of the cover layer 38 made of fluororubber while parameters of Table 1 are satisfied, the strain of the polyparaxylene resin layer 40 is about 2.1% and is suppressed so as to be smaller than 3% that is the yield strain of a polyparaxylene resin. Accordingly, it may be possible to maintain the elasticity of the outer cover member 36.

As described above, the endoscope flexible section 30 according to this exemplary embodiment is provided with the cover layer 38 made of fluororubber. Therefore, it may be possible to provide the outer cover member 36 that has the resistance against high temperature-and-pressure steam (resistance during the sterilization of an autoclave). Further, since the outer cover member 36 is formed by patterning the polyparaxylene resin layer 40 on the outer periphery of the cover layer 38, it may be possible to increase the tear strength and bending rigidity of the endoscope flexible section 30. Furthermore, when the endoscope flexible section 30 is curved, the outer cover member 36 of the endoscope flexible section 30 is required to resist the strain of about 20%. However, in the above mentioned pattern of the polyparaxylene resin layer 40, the strain of the polyparaxylene resin layer 40 may be suppressed to be about 3% or less.

In addition, it is possible to adjust the bending rigidity of the endoscope flexible section 30 to a desired value by changing the density or pitch of the pattern of the polyparaxylene resin layer 40. Further, by changing the density or pitch of the pattern of the polyparaxylene resin layer 40 in accordance with position of the endoscope flexible section in the longitudinal direction (axial direction),

the value of the bending rigidity of the endoscope flexible section 30 can be changed into a desired value at the portion in the longitudinal direction. For example, the bending rigidity of the portion of the flexible tube portion 12A, which is near the main body operation section 14, may be increased in order to obtain thrust when the flexible tube portion is inserted into the body cavity or the like. Further, for example, the bending rigidity of the portion of the flexible tube portion 12A, which is near the angle portion 12B, may be decreased in comparison with the bending rigidity of the portion of the flexible tube portion that is near the main body operation section 14 so that the portion of the flexible tube portion near the angle portion 12B follows the shape of the curved angle portion 12B to some extent when the angle portion 12B is curved. It may be achieved by differing density or pitch of the pattern of the polyparaxylene resin layer 40 between the portion of the flexible tube portion 12A which is near the angle portion 12B and the portion of the flexible tube portion 12A which is near the main body operation section 14 (for example, in FIG. 5, the intervals between the polyparaxylene resin layers 40 are different at the portion of the flexible tube portion 12A which is near the angle portion 12B and the portion of the flexible tube portion 12A which is near the main body operation section 14).

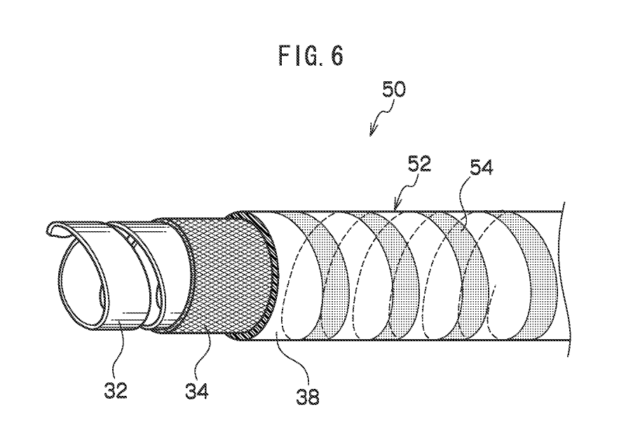

FIG. 6 is a perspective view of an endoscope flexible section used for an endoscope according to a second exemplary embodiment of the invention. Meanwhile, the same members as those of the first exemplary embodiment are denoted by the same reference numerals, and the description thereof will not be repeated.

As shown in FIG. 6, an outer cover member 52 of an endoscope flexible section 50 includes a cover layer 38 that is made of fluororubber, and a polyparaxylene resin layer 54 that is continuously disposed on the outer periphery of the cover layer 38 in pattern of spiral (spirally disposed on the outer periphery of the cover layer 38 in the longitudinal direction). Even in this exemplary embodiment, it may be possible to adjust the bending rigidity of the endoscope flexible section 50 to a desired value by changing the density or pitch of the pattern of the polyparaxylene resin layer 54. Further, by changing the density or pitch of the pattern of the polyparaxylene resin layer 54 in accordance with position of the endoscope flexible section 50 in the longitudinal direction (axial direction), the value of the bending rigidity of the endoscope flexible section 50 can be changed into a desired value at the portion in the longitudinal direction.

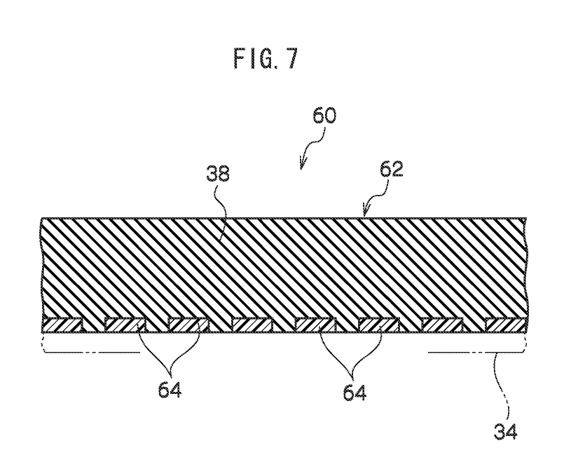

FIG. 7 is a perspective view of an endoscope flexible section used for an endoscope according to a third exemplary embodiment of the invention. Meanwhile, the same members as those of the first and second exemplary embodiments are denoted by the same reference numerals, and the description thereof will not be repeated.

As shown in FIG. 7, an outer cover member 62 of an endoscope flexible section 60 includes a polyparaxylene resin layer 64 and a cover layer 38. The polyparaxylene resin layer 64 is formed on the outer periphery of the net-like tube 34 in pattern such that plural rings along a circumferential direction of the net-like tube 34 are formed. The cover layer 38 is made of fluororubber and is formed on the outer peripheries of the net-like tube 34 and the polyparaxylene resin layer 64. That is, the polyparaxylene resin layer 64 is provided and patterned inside the cover layer 38. Like in the first exemplary embodiment, the endoscope flexible section 60 can also have the resistance against high temperature-and-pressure steam (resistance during the sterilization of an autoclave), and the bending rigidity of the endoscope flexible section can be increased.

Meanwhile, in the endoscope flexible sections according to the first to third exemplary embodiments, fluororubber is used as the material of the cover layer 38 of the outer cover member. However, silicone rubber having the resistance against high temperature-and-pressure steam (resistance during the sterilization of an autoclave) may be used instead of fluororubber.

Meanwhile, in the first to third exemplary embodiments, the polyparaxylene resin layer is formed in a predetermined pattern on the outer periphery or the inside of the cover layer 38. However, the pattern of the polyparaxylene resin layer is not limited thereto. If being discontinuous in the longitudinal direction, other patterns may be formed.

What is claimed is:

1. An endoscope flexible section comprising:
a longitudinal tubular body; and
an outer covering member formed on an outer periphery of the tubular body, the outer covering member including:
a cover layer that covers the outer periphery of the tubular body and is made of fluororubber or silicone rubber; and
a polyparaxylene resin layer formed in a pattern, disposed on an outer periphery of the cover layer or disposed inside the cover layer, so as to be discontinuous in a longitudinal direction of the outer covering member.

2. The endoscope flexible section of claim 1, wherein the pattern of the polyparaxylene resin layer is a plurality of rings, each formed in a circumferential direction of the outer covering member, that are arranged in the longitudinal direction of the outer covering member.

3. The endoscope flexible section of claim 1, wherein the pattern of the polyparaxylene resin layer is a spiral formed along the longitudinal direction of the outer covering member.

4. The endoscope flexible section of claim 1, wherein a bending rigidity of the outer covering member is changed in the longitudinal direction by changing a density or a pitch of the pattern of the polyparaxylene resin layer in the longitudinal direction.

5. The endoscope flexible section of claim 2, wherein a bending rigidity of the outer covering member is changed in the longitudinal direction by changing a pitch of the rings of the polyparaxylene resin layer in the longitudinal direction.

6. The endoscope flexible section of claim 3, wherein a bending rigidity of the outer covering member is changed in the longitudinal direction by changing a pitch of the spiral of the polyparaxylene resin layer in the longitudinal direction.

7. The endoscope flexible section of claim 1, wherein the thickness of the polyparaxylene resin layer is from 5 μm to 80 μm.

8. The endoscope flexible section of claim 2, wherein the thickness of the polyparaxylene resin layer is from 5 μm to 80 μm.

9. The endoscope flexible section of claim 3, wherein the thickness of the polyparaxylene resin layer is from 5 μm to 80 μm.

10. The endoscope flexible section of claim 1, wherein the tubular body includes a spiral tube that is formed by winding a belt-like member in a spiral shape, and a net-like tube that is provided on the outer periphery of the spiral tube and formed in an annular shape by braiding thin wires.

11. The endoscope flexible section of claim 4, wherein a density or a pitch of the pattern of the polyparaxylene resin layer at one end side in the longitudinal direction of the outer covering member is different from that at another side.

12. An endoscope comprising a long insertion section to be inserted into a patient's body cavity comprising an endoscope flexible section including:
a longitudinal tubular body; and
an outer covering member formed on an outer periphery of the tubular body, the outer covering member including:
a cover layer that covers the outer periphery of the tubular body and is made of fluororubber or silicone rubber; and
a polyparaxylene resin layer formed in a pattern, disposed on an outer periphery of the cover layer or disposed inside the cover layer, so as to be discontinuous in a longitudinal direction of the outer covering member.
